(19) United States
(12) Patent Application Publication
(10) Pub. No.: US 2023/0368239 A1
(43) Pub. Date: Nov. 16, 2023

(54) SYSTEM AND METHOD FOR SENSOR-BASED MASK DETECTION

(71) Applicant: Wells Fargo Bank, N.A., San Francisco, CA (US)

(72) Inventor: Brian J. Jacobsen, Elm Grove, WI (US)

(21) Appl. No.: 17/247,105

(22) Filed: Nov. 30, 2020

(51) Int. Cl. G06Q 30/02; G06K 9/00; G08B 21/18; H04W 4/02; H04L 29/08; H04Q 9/00; G06K 7/10; G06F 16/903; G01N 33/497; G01K 13/00; G01F 15/00

(52) U.S. Cl. CPC G06Q 30/0234; G06K 9/00228; G08B 21/182; H04W 4/023; H04L 67/306; H04Q 9/00; G06K 7/10297; G06K 7/10386; G06K 7/10396; G06F 16/90335; G01N 33/497; G01K 13/00; G01F 15/00; H04W 4/80

(57) ABSTRACT

Various examples are directed to computer-implemented systems and methods for sensor-based mask detection. A method includes communicating with a sensor attached to or embedded in a face mask to determine whether a user is in proximity to the face mask. An alert is provided based on a determination that the face mask is not in proximity to the user. The method includes communicating with the sensor to determine whether the user is wearing the face mask, transmitting a request to determine a location of the user, and querying a datastore to determine whether the location is affiliated with an entity that offers rewards to users wearing face masks. A user profile is updated to indicate a reward is available to the user based on a determination that the user is wearing the face mask at the location of the entity that offers rewards to users wearing face masks.

### CROSS REFERENCE TO RELATED APPLICATIONS

This application is related to co-pending, commonly assigned U.S. patent application Ser. No. ______, entitled “ENFORCING SOCIAL DISTANCING USING TECHNOLOGY” (Attorney Docket No. 4423.377US1), and related to co-pending, commonly assigned U.S. patent application Ser. No. ______, entitled “SYSTEM AND METHOD FOR SENSOR-BASED SOCIAL DISTANCING” (Attorney Docket No. 4423.378US1), both filed on even date herewith, which are hereby incorporated by reference herein in their entirety.

### BACKGROUND

In view of the proliferation of contagious airborne viruses, the wearing of a face mask or face covering has significant importance. Some experts have opined that the proper use of a face mask is the one of the most effective tools in limiting the spread of Covid-19 and other airborne viruses. Some people find it difficult to remember to bring their face masks with them. Others have difficulty properly wearing their face masks.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not of limitation, in the figures of the accompanying drawings, in which:

FIG. 1 illustrates an example embodiment of a computer system for use in the present subject matter;

FIG. 2 illustrates an exemplary infrastructure for use in the present subject matter;

FIG. 3A illustrates an example embodiment of a method for sensor-based mask detection, according to various embodiments;

FIG. 3B illustrates an example embodiment of a method for detection of mask-wearing, according to various embodiments;

FIGS. 4A-4C illustrate example embodiments of sensors for masks and premises, according to various embodiments; and

FIG. 5 is a block diagram of a machine in the example form of a computer system within which a set of instructions may be executed, for causing the machine to perform any one or more of the methodologies discussed herein.

### DETAILED DESCRIPTION

The present subject matter provides systems and methods for sensor-based mask detection. In one embodiment, the present subject matter is included in an application on a person's smartphone. The application uses the geolocation electronics in the phone to determine when the person is leaving home, triggering the mask check in various embodiments. The application then sends a signal, such as a ping, to a sensor on the mask to determine if the person (i.e., the user) has the mask on their person as they are leaving home. If the mask is not with the user, the application can provide a notification to the user that the user has forgot the mask.

In various embodiments, the present subject matter can detect not only if the person remembered their mask, it can also detect if the person is wearing the mask. A sensor in or on the mask or photographic technology can be used to determine if the person is wearing the mask. In various examples, the user can get rewards such as points, credit card benefits, or discounts at participating retailers for remembering to have their mask on their person. In addition, the user can obtain the rewards (or additional rewards) if wearing the mask in various embodiments.

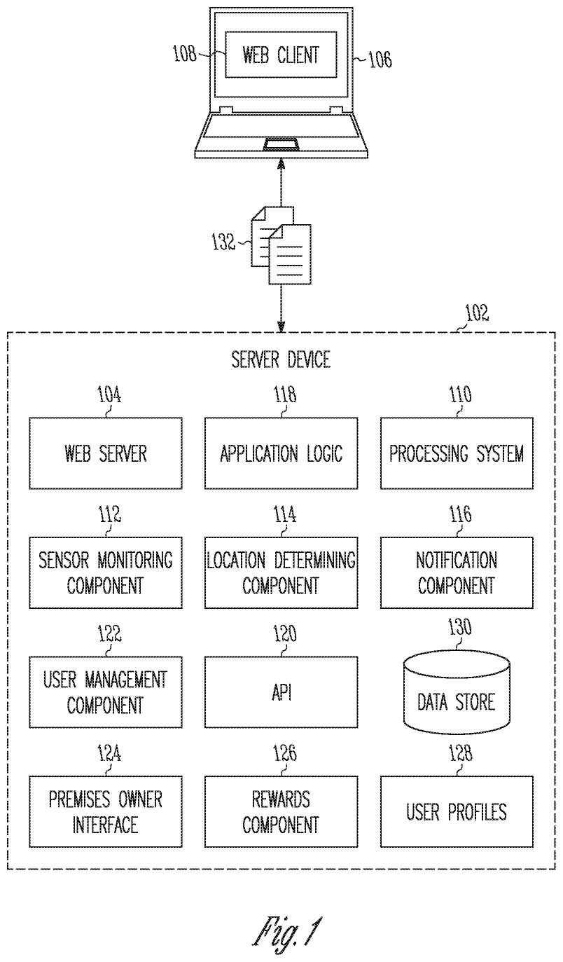

FIG. 1 illustrates an example embodiment of a computer system for use in the present subject matter. FIG. 1 comprises a server device 102, web server 104, client device 106, web client 108, processing system 110, sensor monitoring component 112, location determining component 114, notification component 116, application logic 118, application programming interface (API) 120, user management component 122, premises owner interface 124, rewards component 126, user profiles 128, data store 130, and data 132.

Client device 106 may be a computing device which may be, but is not limited to, a smartphone, tablet, laptop, multi-processor system, microprocessor-based or programmable consumer electronics, game console, set-top box, or other device that a user utilizes to communicate over a network. In various examples, a computing device includes a display module (not shown) to display information (e.g., in the form of specially configured user interfaces). In some embodiments, computing devices may comprise one or more of a touch screen, camera, keyboard, microphone, or Global Positioning System (GPS) device. Client device 106 may be associated with one or more entities that interact with server device 102. An entity may be an individual, group of individuals, or company in various examples.

Client device 106 and Server device 102 may communicate via a network (not shown). The network may include local-area networks (LAN), wide-area networks (WAN), wireless networks (e.g., 802.11 or cellular network), the Public Switched Telephone Network (PSTN) Network, ad hoc networks, cellular, personal area networks or peer-to-peer (e.g., Bluetooth®, Wi-Fi Direct), or other combinations or permutations of network protocols and network types. The network may include a single Local Area Network (LAN) or Wide-Area Network (WAN), or combinations of LAN's or WAN's, such as the Internet.

Client device 106 and server device 102 may communicate data 132 over the network. Data 132 may be, but is not limited to, search requests, search results, market offers (as discussed in more detail below), user information, validation results, and decision results.

In some examples, the communication may occur using an application programming interface (API) such as API 120. An API provides a method for computing processes to exchange data. A web-based API (e.g., API 120) may permit communications between two or more computing devices such as a client and a server.

Server device 102 is illustrated as set of separate elements (e.g., component, logic, etc.). However, the functionality of individual elements may be performed by a single element. An element may represent computer program code that is executable by processing system 110. The program code may be stored on a storage device (e.g., data store 130) and loaded into a memory of the processing system 110 for execution. Portions of the program code may be executed in a parallel across multiple processing units (e.g., a core of a general-purpose computer processor, a graphical processing unit, an application specific integrated circuit, etc.) of processing system 110. Execution of the code may be performed on a single device or distributed across multiple devices. In some example, the program code may be executed on a cloud platform (e.g., MICROSOFT AZURE® and AMAZON EC2®) using shared computing infrastructure.

Server device 102 may include web server 104 to enable data exchanges with client device 106 via web client 108. Although generally discussed in the context of delivering webpages via the Hypertext Transfer Protocol (HTTP), other network protocols may be utilized by web server 104 (e.g., File Transfer Protocol, Telnet, Secure Shell, etc.). A user may enter in a uniform resource identifier (URI) into web client 108 (e.g., the INTERNET EXPLORER® web browser by Microsoft Corporation or SAFARI® web browser by Apple Inc.) that corresponds to the logical location (e.g., an Internet Protocol address) of web server 104. In response, web server 104 may transmit a web page that is rendered on a display device of a client device (e.g., a mobile phone, desktop computer, etc.).

Additionally, web server 104 may enable a user to interact with one or more web applications provided in a transmitted web page. A web application may provide user interface (UI) components that are rendered on a display device of client device 106. The user may interact (e.g., select, move, enter text into) with the UI components, and based on the interaction, the web application may update one or more portions of the web page. A web application may be executed in whole, or in part, locally on client device 106. The web application may populate the UI components with data from external sources or internal sources (e.g., data store 130) in various examples.

For example, server device 102 may provide a web application (e.g., user management component 122) to a user to provide an interface for the user to interact with the application. Data store 130 may store user information and/or rewards information, including unique identifiers to identify users and face masks. That is, in the various examples described herein, an application may provide by the server 102 that permits a user (e.g., via the client device 106) to receive, view, access, and redeem rewards. As further described herein, rewards may be offered for activity related to carrying a face mask and/or wearing a face mask. Further, the application provided by the server 102 may provide one or more alerts. For instance, alerts may also be related to carrying a face mask and/or wearing a face mask. In certain examples, the application may also facilitate communication with one more sensors attached to or embedded in a face mask. For instance, this may be facilitated using integrated technology of the client device 106, such as wireless communication technology of a user device (e.g., a smartphone).

The web application may be executed according to application logic 118. Application logic 118 may use the various elements of server device 102 to implement the web application. For example, application logic 118 may issue API calls to retrieve or store data from data store 130 and transmit it for display on client device 106. Similarly, data entered by a user into a UI component may be transmitted using API 120 back to the web server 104.

Data store 130 is depicted as singular element but may in actuality be multiple data stores. The specific storage layout and model used in by data store 130 may take a number of forms—indeed, a data store 130 may utilize multiple models. Data store 130 may be, but is not limited to, a relational database (e.g., SQL), non-relational database (NoSQL) a flat file database, object model, document details model, graph database, shared ledger (e.g., blockchain), or a file system hierarchy. Data store 130 may store data on one or more storage devices (e.g., a hard disk, random access memory (RAM), etc.). The storage devices may be in standalone arrays, part of one or more servers, and may be located in one or more geographic areas.

User profiles 128 may store the profiles of users that interact with server device 102. A user profile may include a user identifier (e.g., a username) and a password. A user profile may also include a user's name, address, phone number, account numbers, unique identifiers, and other information.

In various embodiments, the sensor monitoring component 112 determines whether a user has a face mask in proximity to the user, such as carried on the user's person, by executing software (either on the server of a user device) to prompt the user device to communicate with a sensor or sensors attached to or embedded in the user's face mask, or directly communicating with said sensor or sensors. The location determining component 114 can determine if the user is departing from their home location, and the notification component 116 can send an alert to a user device if the user does not have a face mask in proximity to the user, in various embodiments. The sensor monitoring component 112 may further determine whether the user is wearing the face mask by communicating with the sensor or sensors, or by analyzing a photographic image of the user.

In various examples, the location determining component 114 may further determine a location of the user to determine if the user is located at a specific geographic location (e.g., business premises) corresponding to a retail establishment, or other entity. For instance, the rewards component 126 can determine whether the specific geographic location is affiliated with an entity that supplies rewards to users wearing face masks, and can provide rewards to the user when the user is wearing the face mask at the affiliated premises. The present subject matter may further provide internal tools, such as a look-up tool or premises owner interface 124, which may enable premises owner employees to review and update available rewards offers and statuses. For instance, an application may be provided by the server 102 that permits a retail user (e.g., via the client device 106) to offer and update available rewards. That is, the application may provide one or more user interfaces that permit a retail user to define a rewards offer, and the conditions associated with that rewards offer. Satisfaction of the conditions may result in the reward being delivered. For example, the conditions associated with a rewards offer may be associated with a potential customer wearing a mask when a location of that potential customer corresponds to a geographic location of a retail establishment of the retail user.

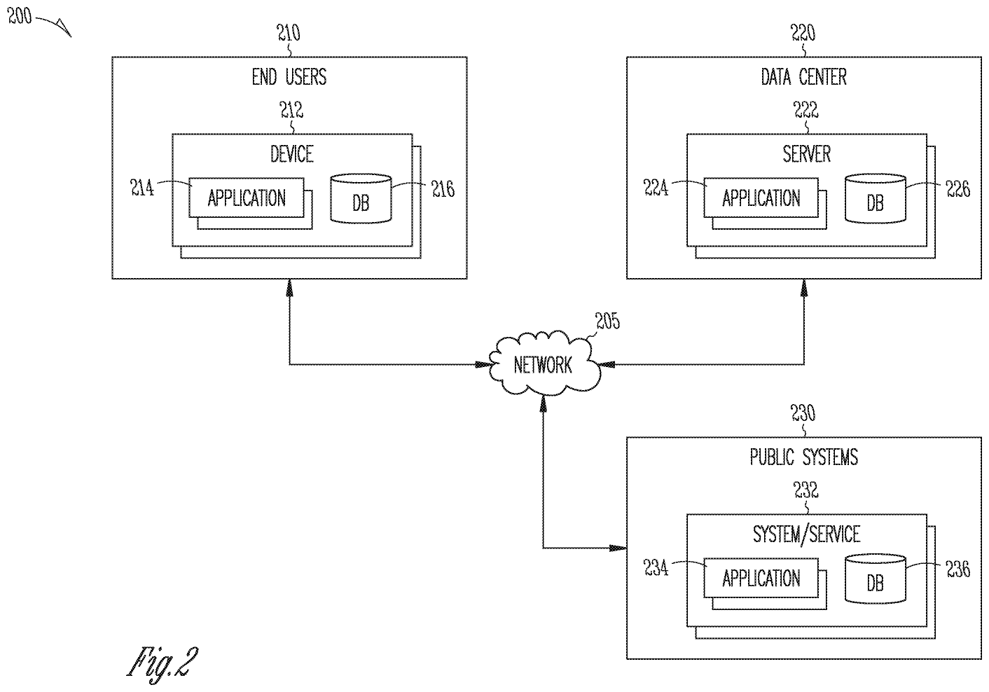

FIG. 2 illustrates an exemplary infrastructure for providing the system of the present subject matter. The infrastructure may comprise a distributed system 200 that may include a client-server architecture or cloud computing system. Distributed system 200 may have one or more end users 210. An end user 210 may have various computing devices 212, which may be a machine 500 as described below. The end-user computing devices 212 may comprise applications 214 that are either designed to execute in a stand-alone manner, or interact with other applications 214 located on the device 212 or accessible via the network 205. These devices 212 may also comprise a data store 216 that holds data locally, the data being potentially accessible by the local applications 214 or by remote applications.

The system 200 may also include one or more data centers 220. A data center 220 may be a server 222 or the like associated with a business entity that an end user 210 may interact with. The business entity may be a computer service provider, as may be the case for a cloud services provider, or it may be a consumer product or service provider, such as a retailer. The data center 220 may comprise one or more applications 224 and databases 226 that are designed to interface with the applications 214 and databases 216 of end-user devices 212. Data centers 220 may represent facilities in different geographic locations where the servers 222 may be located. Each of the servers 222 may be in the form of a machine(s) 500.

The system 200 may also include publicly available systems 230 that comprise various systems or services 232, including applications 234 and their respective databases 236. Such applications 234 may include news and other information feeds, search engines, social media applications, and the like. The systems or services 232 may be provided as comprising a machine(s) 500.

The end-user devices 212, data center servers 222, and public systems or services 232 may be configured to connect with each other via the network 205, and access to the network by machines may be made via a common connection point or different connection points, e.g. a wireless connection point and a wired connection. Any combination of common or different connections points may be present, and any combination of wired and wireless connection points may be present as well. The network 205, end users 210, data centers 220, and public systems 230 may include network hardware such as routers, switches, load balancers and/or other network devices.

Other implementations of the system 200 are also possible. For example, devices other than the client devices 212 and servers 222 shown may be included in the system 200. In an implementation, one or more additional servers may operate as a cloud infrastructure control, from which servers and/or clients of the cloud infrastructure are monitored, controlled and/or configured. For example, some or all of the techniques described herein may operate on these cloud infrastructure control servers. Alternatively, or in addition, some or all of the techniques described herein may operate on the servers 222.

The present subject matter provides incentives for a person to remember and wear their face mask. In one embodiment, a sensor is embedded in or attached to a face mask. The sensor may include a Bluetooth transmitter (or other wireless communication transmitter, receiver, or transceiver) or radio frequency identification (RFID) chip that communicates with a user device, such as a person's smartphone. The sensor may be a latent device that remains dormant until receiving a signal from the smartphone, or other device waking-up the sensor. Once a signal is received, the sensor responds to the signal, which may include transmitting a response to the smartphone. The sensor, which in some examples may be a proximity sensor, may be an off the shelf Bluetooth (or other wireless) transmitter or RFID chip.

While in some examples, the sensor embedded in or attached to the face mask may communicate via discrete signals, in various other examples, a continuous stream of communication may be established between the sensor and the user device (e.g., the user's smartphone). Accordingly, in some examples, the person may be periodically “checked” to ascertain if the person is proximate face mask, or if the person is consistently wearing the face mask over a given period of time. The period of time (e.g., frequency of polling) may be one minute, five minutes, or thirty minutes, to name a few examples. In this manner, the system can determine if the person has their mask with them, and is keeping the mask in their proximity.

In one embodiment, the present subject matter is included in an application on a user device such as a person's smartphone, or included in an application accessed through a web browser using the person's smart phone. The application 214 uses the geolocation electronics in the phone to determine when the person is leaving home, triggering the mask check in various embodiments. For instance, when the application determines that a geolocation of the smartphone is not within a geographic range corresponding to the user's home, the application may send a signal, such as a ping, to the mask to determine if the person has the mask in their proximity as they are leaving their home.

In some examples, the signal to the mask may be received by the sensor attached to or embedded in the mask and elicit a response from the sensor embedded in the mask. For instance, the response may be sent from the sensor if the mask is within a desired proximity (or in proximity) to the smartphone. In one example, the desired proximity between the mask and the phone may be the range of communication of the sensor embedded or attached to the mask. In other examples, proximity or desired proximity refers to the mask being within a programmable or predetermined distance from either the user or the user device. Receipt of the response from the sensor of the mask confirms that the mask is proximate the smart phone, and the user. As discussed, this may be repeated dynamically or continually over a period of time to ensure that the user is keeping the mask on their person. If a response is not received from the sensor of the mask, the application can provide a notification to the user that the user has forgot the mask. While described herein as a signal to the sensor from the phone, and a response from the sensor to the smartphone, in various other examples a signal may be sent from the sensor to the smartphone. That is, the sensor may broadcast a beacon, the detection of which (e.g., by the phone) may confirm that the person has the mask in their proximity.

In various embodiments, the user can synch or register a number of masks with the application, so the smartphone knows which masks to search for when determining if the person has their mask with them. In one embodiment, the mask may have a quick response (QR) code or other scannable feature to register the mask with the application. For instance, each mask may have a unique identification code, or unique QR code. The user can register the masks using a web application using a user interface provided by the web server 104 in FIG. 1, for example. In some embodiments, a batch of masks or a box of masks could all have the same code, so that a user can synch the whole box of masks by using a scannable feature on the box. In some embodiments, the sensor or sensors can clip on the mask, so that a person can use any existing mask with the application of the present subject matter. The sensor can be in the form of a pendant or charm that physically or electrically attaches to the mask, in some embodiments.

The user can get reward points, credit card benefits, or discounts at participating retailers for remembering their mask using the rewards component 126, in some embodiments. In addition, the user can obtain rewards if wearing the mask.

In various examples, the present subject matter can detect not only if the person remembered their mask, it can also detect if the person is wearing the mask. In one embodiment, if the person is wearing smart glasses (such as Google glasses) or other smart headwear, the smart headwear can detect the proximity of the sensor in the mask within a small distance (1 to 3 inches, for example) to determine of the person is wearing the mask. In some embodiments, a person can take a self photograph (or “selfie”) and the sensor monitoring component 112 of the present application can determine if the person is wearing the mask by analyzing the selfie using image processing technology. In some embodiments, the camera of the smartphone can detect a pattern on the mask to determine if the person is wearing the mask. In various embodiments, the application searches an image below the person's eyeballs in attempt to detect the mask.

In various embodiments, the mask includes a sensor or sensors to detect if the person is wearing the mask, which can then be communicated from the mask to the person's smartphone. For instance, this sensor or sensors may be the same sensor or sensors that communicates with the smart phone of the person to determine if the mask is proximate the person when the person is leaving their home. In some embodiments, the sensor included with the mask is a moisture sensor that detects a person's breath to determine if the mask is being worn. For example, if a moisture level detected by the moisture sensor exceeds a moisture threshold, it may be determined that the person is wearing the mask. In some embodiments, the sensor included with the mask is an airflow sensor that detects a person's breath to determine if the mask is being worn. That is, if an airflow level detected by the airflow sensor exceeds an airflow threshold, it may be determined that the person is wearing the mask. In some other embodiments, the sensor included with the mask is a temperature sensor that detects a person's body heat to determine if the mask is being worn. That is, if a temperature detected by the temperature sensor exceeds temperature threshold, it may be determined that the person is wearing the mask. In some embodiments, the sensor, such as a piezoelectric sensor, is configured to determine if the mask is folded (and thus not being worn) or in an extended configuration (consistent with being worn). For instance, the piezoelectric sensor may generate an electrical charge (e.g., signal) as a result of mechanical stress on the mask, such as an unfolding or stretching of the mask. The sensor or sensors can communicate with the sensor monitoring component 112 to determine the position of the mask, or the shape of the mask currently, to determine if the mask is being worn. Other sensors can be used without departing from the scope of the present subject matter.

In some embodiments, a plurality of sensors on the mask (or attached to the mask) can determine if the mask is being worn based on skin conductivity, by attempting to conduct a signal between the sensors. For example, two or more sensors on a nose bridge of the mask may detect whether the mask is positioned over the bridge of the nose and cheeks, thus determining whether the mask is being worn properly. In a similar manner, the sensors on the mask can determine whether the mask is hooked over the person's ears, such as by using a sensor on or near one or more elastic bands of the mask, to determine whether the mask is being worn properly. The sensors may sense oil of the wearer's skin, or secretions, to determine of the mask is being worn, in some embodiments.

While described in some examples as including a single sensor, in various other embodiments a face mask may include a plurality of sensors. Each sensor of the plurality may be the same type of sensor, or a different type of sensor. For example, the face mask may include a first sensor of a first type (e.g., a temperature sensor), and a second sensor of second type (e.g., an airflow sensor). A combination of different sensor types offers the benefit of improved data reporting as it offers a more comprehensive report of collected information. While in one example, the first sensor may be used to help determine if the user has the face mask in their proximity, and the second sensor may be used to help determine if the face mask is being worn, in various other examples some or all of the plurality of sensors may be used for both purposes.

As previously discussed herein, in various examples the sensor or sensors attached or embedded in the mask may be continuously or dynamically polled to determine if the mask is being worn by the person over an extended period of time. In certain other examples, the sensor or sensors may continuously or periodically transmit a signal (e.g., a beacon) when the sensor or sensors are sensing information, e.g., a moisture level, an airflow level, a temperature, an electrical charge, a conductivity, etc. Such a beacon may be located and received by the person's smartphone, for instance, when that person has left their home. A continuous or dynamic communication stream with the sensors ensures that the person is consistently wearing their mask, and not periodically taking the mask off, or re-positioning the face mask to an improper position.

The application of the present subject matter can provide rewards using rewards component 126 based on the extent of a person's compliance with remembering and/or wearing a mask, in various embodiments. The rewards may be points that can be redeemed for merchandise or credit, in some embodiments. For example, the application can reward a person if they have their mask with them when they leave their home. In another example, the person can receive rewards if they wear a mask inside a retail establishment. The retailer can participate and provide the rewards, in some embodiments. In further embodiments, a credit card company can participate and provide rewards, such as extra cash back (e.g., 1.1% instead of the normal 1%) for shopping while wearing their mask. A government entity can participate and provide rewards, in some embodiments. The application can provide other positive or negative incentives for mask wearing compliance, in various embodiments. For example, a notification can be provided by notification component 116 on a user device if a mask is not being worn in compliance with local rules, regulations, or retailer requirements, or not being worn properly (e.g., not being worn over the bridge of a user's nose). Likewise, a social media post to user-selected accountability partners (similar to a dieting regime) can be made to let others know if the user is in compliance. This can provide peer pressure as an incentive to the user to comply with the desired behavior.

A participating retailer may provide bonus cash back for compliance by a user in the participating retailer's store or premises, in an embodiment. For example, a user may get a discount at the retailer location for compliance. A participating financial institution may provide a mechanism to round-up purchases and put the difference in a savings account as a reward to the user for compliance using premises owner interface 124, in an embodiment. For example, if the purchase is for $1.60, an additional $0.40 could be transferred from the user's checking to savings as a reward. A similar transfer could be used as a negative incentive to another user, as funds transferred to a savings account are more difficult to access, in an embodiment. In another embodiment, as a negative incentive, the user may be forced to make a fixed donation to a charity, such as the Humane Society, for every time they leave their home or enter an establishment without their mask, or not wearing their mask, or not properly wearing their mask. The aforementioned donation could be an escalating amount for repeat offenders, in an embodiment.

The present application provides a rewards points system using rewards component 126, in various embodiments. The user can get points (similar to airline or credit card points) for compliance, and can redeem the points to purchase goods or services at participating merchants, in various embodiments. A user may receive a number of points scaled to the percentage of compliance of mask remembering and/or mask wearing, in various embodiments. In various examples, compliance with one or more rules, regulations, policies, or retailer requirements for possession of a mask (or wearing of a mask) may be detected and enforced using the techniques discussed herein. Satisfaction of one or more conditions associated with a reward may be determined based on the techniques described herein. If the conditions are satisfied, the reward may be granted.

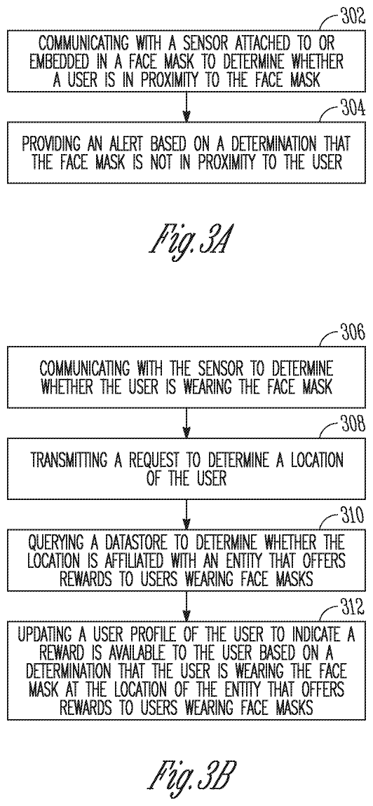

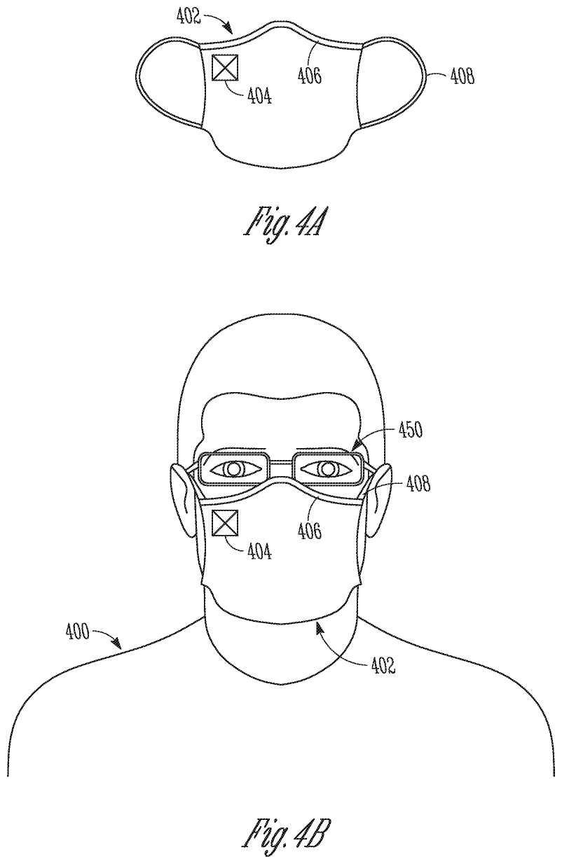

FIG. 3A illustrates an example embodiment of a method for sensor-based mask detection, according to various embodiments. The method includes communicating with a sensor attached to or embedded in a face mask to determine whether a user is in proximity to the face mask at step 302, and sending an alert to a user device if the user does not have a face mask in proximity to the user at step 304. The method can be combined with the method of FIG. 3B in some embodiments. FIG. 3B illustrates an example embodiment of a method for detection of mask-wearing, according to various embodiments. The method includes communicating with the sensor to determine whether the user is wearing the face mask at step 306, transmitting a request to determine a location of the user at step 308, and querying a datastore to determine whether the location is affiliated with an entity that offers rewards to users wearing face masks at step 310. At step 312, user profile of the user is updated to indicate a reward is available to the user when the user is wearing the face mask at the location that supplies rewards to users wearing face masks. The methods of FIGS. 3A and 3B may be performed using the system components described in FIG. 1, in various embodiments.

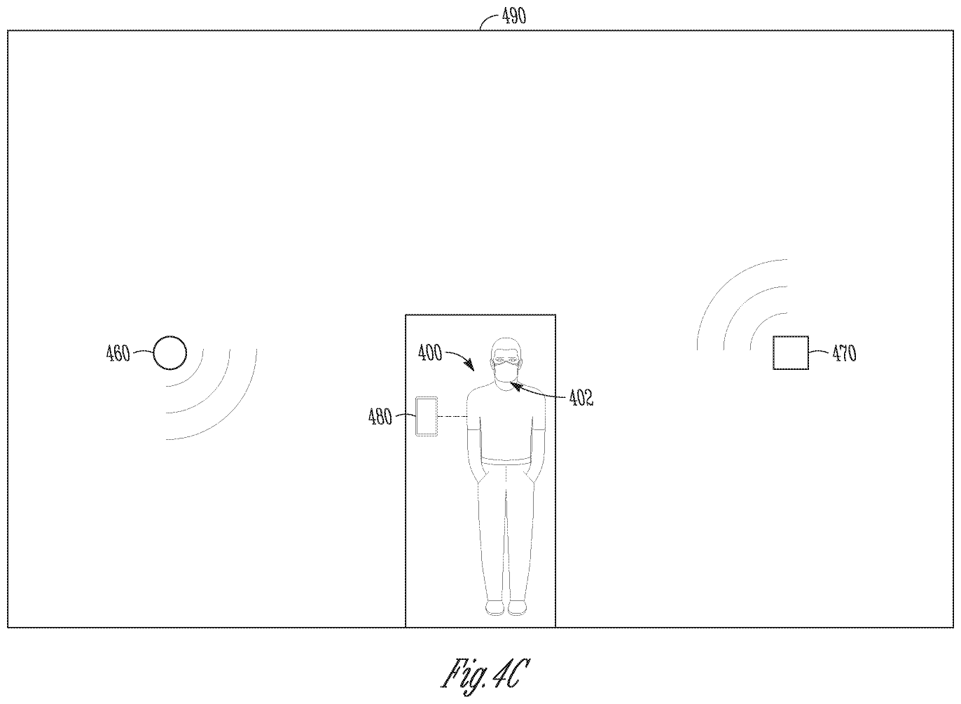

FIGS. 4A-4C illustrate example embodiments of sensors for masks and premises, according to various embodiments. FIG. 4A illustrates an example embodiment of a mask 402 with an attached or embedded sensor 404. A single sensor 404 is shown, but multiple sensors of the same or different types may be used in various embodiments. FIG. 4B illustrates an example embodiment of a mask 402 as worn by a person 400 (also referred to herein as the “user”, in some examples). FIG. 4C illustrates an example embodiment of use of the present subject matter at a retail premises 490.

According to various embodiments, determining whether a user has a face mask 402 in proximity of the user includes using the user device 480 to detect proximity of a sensor 404 attached to or embedded in the face mask 402. In various embodiments, a programmable distance threshold is selected to determine whether the face mask is being carried with the user. That is, the programmable distance threshold may be compared to a distance between the face mask 402 and the user 400, or between the face mask 402 and the user device 480. If the programmable distance is exceeded, the face mask 402 is determined to not be proximate the user. In some embodiments, the sensor 404 includes one or more of a Bluetooth device or a near-field communication (NFC) device. In some embodiments, the sensor 404 includes one or more of a temperature sensor configured to detect body heat of a user, a moisture sensor configured to detect moisture in a breath from the user, or an airflow detector configured to detect the breath from the user. Other types of sensors can be used without departing from the scope of the present subject matter.

Determining whether the user 400 is wearing the face mask 402 includes using a signal received from a sensor 404 attached to or embedded in the face mask, in various embodiments. The sensor 404 can be connected, either physically or electrically, or both, to a nose portion 406 of the mask to determine if the mask 402 is being worn, in an embodiment. The mask 402 may include elastic headband and/or elastic ear bands 408 or other means of attachment to the user 400, in various embodiments. According to various embodiments, determining whether the user 400 is wearing the face mask 402 includes using a wearable device such as smart glasses 450 or a smart pendent to detect proximity of the face mask 402.

Sending an alert to a user device includes sending one or more of an audible alert (such as notification tone or spoken words from a virtual assistant), text alert or a vibration alert to the user device to remind the user to bring the face mask, according to various embodiments. In some embodiments, determining whether the user is wearing the face mask includes: directing the user to take a photograph (with the user device 480 such as a smartphone or wearable device), receiving the photograph from the user, and analyzing the photograph to determine whether the face mask is on a user's face.

In various embodiments, determining location of the user 400 includes communication with a beacon 460 and/or a wireless router 470 at the location, such as a retail premises 490. For example, the user device 480 may communicate with the beacon 460 and/or wireless router 470 by sending or receiving a signal from the beacon 460 and/or wireless router 470 to assist in location determination. According to various embodiments, providing rewards to the user when the user is wearing the face mask includes providing points to the user, providing cash back from a purchase to the user and/or providing a discount on a purchase to the user. In some embodiments, rewards are provided to the user when the user has a face mask in proximity to the user regardless if it is being worn. Determining whether the location is affiliated with an entity that supplies rewards to users wearing face masks includes using a look-up table of participating entities, in some embodiments.

Various embodiments include a system including a computing system comprising one or more processors and a data storage system in communication with the one or more processors, where the data storage system comprises instructions thereon that, when executed by the one or more processors, causes the one or more processors to execute methods of the present subject matter. Various embodiments include a non-transitory computer-readable storage medium, the non-transitory computer-readable storage medium including instructions that, when executed by computers, cause the computers to perform operations to execute methods of the present subject matter.

Various embodiments of the present subject matter generally relate to providing rewards when a person wears a mask. More specifically, the present subject matter determines if a user has a face mask and determines whether or not a user is wearing a facemask, by using a facemask that has the functionality to communicate with a user device, or any type of device. In some embodiments, Bluetooth communication between a user device and a sensor on or in the mask is used to determine if a user has a face mask and whether or not the person is wearing the face mask. Also, the present subject matter determines when the person has or is wearing the face mask, determines the location of the user and if the location provides rewards to a user based on wearing a mask.

In various embodiments, a facemask is embedded with a Bluetooth transceiver that emits a signal received at a user's cellphone. When a user enters a retail establishment, such as a store, the user's cellphone receives the signal, which may be indicative of the user at least having the mask, i.e., in their pocket. Based on receiving the signal, rewards may be provided to the user. For example, a user may get cash back on their credit card or rewards, i.e., store cash, that are redeemable at the retail establishment. If the user can establish that they are wearing the mask, such as taking a picture, e.g., a selfie, the user may be awarded more rewards, in various embodiments. Also, if the user is at home and their cellphone is not picking up a signal from the facemask, i.e., the user is not wearing the face mask or is not carrying the face mask, the user's cellphone may send an alert to the user when a determination is made that the user is about to leave their home.

Other mechanisms may be used to determine if the user is actually wearing the mask besides taking a picture. For example, Google Glass, glasses, earrings, necklace/chain/pendant, an attachment to any of these with a sensor such as a Bluetooth receiver or transmitter or transceiver (or other wearable) can detect when the mask is in very close proximity to the face. In some embodiments, the facemask may include breath/heat/moisture detecting mesh that can detect if the mask is covering the user's mouth.

In various embodiments, the provided rewards may include points (cash-back, discount, other standard points options) from participating sponsoring organizations (e.g., retailer where user is located, local or national government, etc.) that can determine framework for points allocation based on a desired strength of the mask-wearing incentive. According to various embodiments, the present subject matter may identify who the sponsoring organization is based on geolocation, QR-code scan at entrance to location, or hand-entry of an identifier associated with a location. Other provided rewards may include a sweepstakes entry or a charitable donation, in various embodiments.

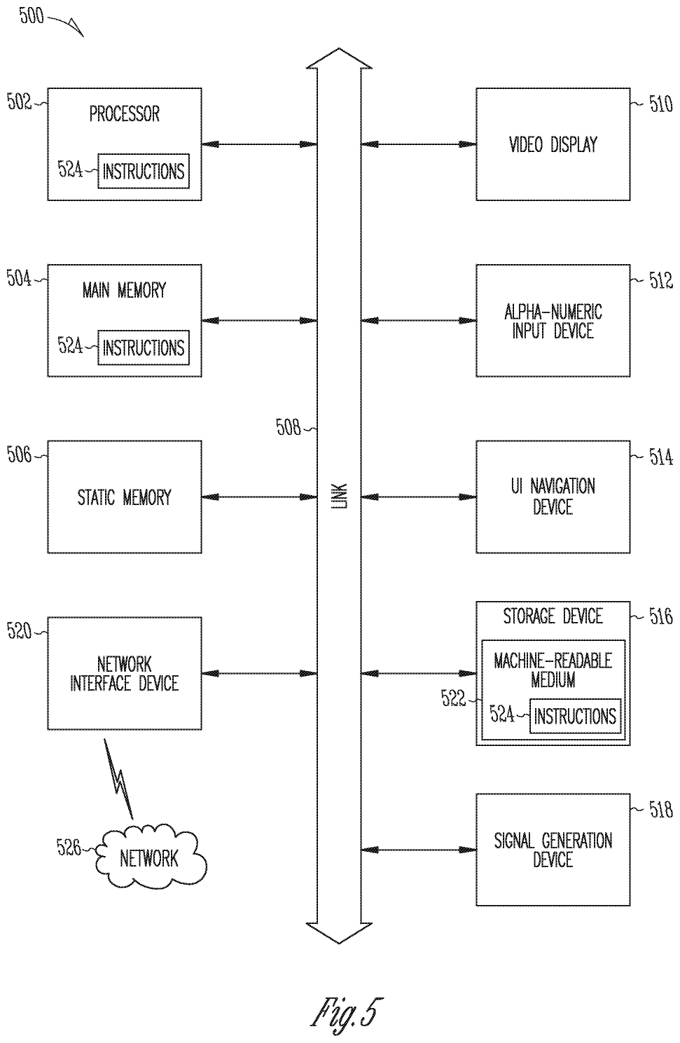

FIG. 5 is a block diagram illustrating a machine in the example form of a computer system 500, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a set-top box (SIB), a personal digital assistant (PDA), a mobile or cellular telephone such as a smartphone, a wearable device such as a smart watch, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

Example computer system 500 includes at least one processor 502 (e.g., a central processing unit (CPU), a graphics processing unit (GPU) or both, processor cores, compute nodes, etc.), a main memory 504 and a static memory 506, which communicate with each other via a link 508 (e.g., bus). The computer system 500 may further include a video display unit 510, an alphanumeric input device 512 (e.g., a keyboard), and a user interface (UI) navigation device 514 (e.g., a mouse). In one embodiment, the video display unit 510, input device 512 and UI navigation device 514 are incorporated into a touch screen display. The computer system 500 may additionally include a storage device 516 (e.g., a drive unit), a signal generation device 518 (e.g., a speaker), a network interface device 520, and one or more sensors (not shown), such as a global positioning system (GPS) sensor, compass, accelerometer, or other sensor.

The data storage device 516 includes a machine-readable medium 522 on which is stored one or more sets of data structures and instructions 524 (e.g., software) embodying or utilized by any one or more of the methodologies or functions described herein. The instructions 524 may include a machine learning system or algorithm, and may also reside, completely or at least partially, within the main memory 504, static memory 506, and/or within the processor 502 during execution thereof by the computer system 500, with the main memory 504, static memory 506, and the processor 502 also constituting machine-readable media.

While the non-transitory computer-readable storage medium 522 is illustrated in an example embodiment to be a single medium, the term “machine-readable medium” or “computer-readable medium” may include a single medium or multiple media (e.g., a centralized or distributed database, and/or associated caches and servers) that store the one or more instructions 524. The term “machine-readable medium” shall also be taken to include any tangible medium that is capable of storing, encoding or carrying instructions (e.g., instructions 524) for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including, but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of well-known transfer protocols (e.g., HTTP). Examples of communication networks include a local area network (LAN), a wide area network (WAN), the Internet, mobile telephone networks, plain old telephone system (POTS) networks, and wireless data networks (e.g., Wi-Fi, 3G, and 6G LTE/LTE-A or WiMAX networks). The term “transmission medium” shall be taken to include any intangible medium that is capable of storing, encoding, or carrying instructions for execution by the machine, and includes digital or analog communications signals or other intangible medium to facilitate communication of such software.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure, for example, to comply with 37 C.F.R. § 1.72(b) in the United States of America. It is submitted with the understanding that it will not be used to interpret or limit the scope or meaning of the claims.

Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

1. A computer-implemented method comprising:
detecting, by a computer system, that a user is departing from a home location;
based on the detection, initiating communication, by the computer system, with a face mask, wherein the face mask comprises:
a material configured to cover a mouth and nose of the user;
one or more elastic bands attached to the material; and
a sensor embedded in or attached to the material, the sensor configured to provide a response when queried by the computer system;
determining, by the computer system, whether the user is in proximity to the face mask based on the communication with the sensor;
providing, by the computer system, an alert based on a determination that the face mask is not in proximity to the user upon departing from the home location;
communicating, by the computer system, with the sensor to determine whether the user is wearing the face mask;
transmitting, by the computer system, a request to determine a location of the user;
querying, by the computer system, a datastore to determine whether the location is affiliated with an entity that offers rewards to users wearing face masks; and
automatically updating, by the computer system; a user profile of the user to indicate a reward is available to the user based on a determination that the user is wearing the face mask at the location of the entity that offers rewards to users wearing face masks.

2. The computer-implemented method of claim 1, wherein communicating with the sensor to determine whether the user is in proximity to the face mask includes detecting that the user is departing from the home location of the user based on the location of the user.

3. The computer-implemented method of claim 1, further comprising determining whether the face mask is being carried with the user based on a comparison of a distance between the face mask and the user and a programmable distance threshold.

4. The computer-implemented method of claim 1, wherein the sensor includes one or more of a Bluetooth device or a near-field communication (NFC) device, and wherein communicating with the sensor to determine whether the user is in proximity with face mask includes communicating with the sensor using a user device.

5. The computer-implemented method of claim 4, wherein providing an alert includes sending one or more of an audible alert, a text alert or a vibration alert to the user device.

6. The computer-implemented method of claim 4, wherein the user device includes one or more of a smartphone or a wearable device.

7. The computer-implemented method of claim 1, wherein the sensor includes one or more of a temperature sensor configured to detect body heat of the user, a moisture sensor configured to detect moisture in a breath from the user, or an airflow detector configured to detect airflow from the breath from the user.

8. The computer-implemented method of claim 1, wherein communicating with the sensor to determine whether the user is wearing the face mask includes: directing the user to take a photograph, receiving the photograph from the user, and analyzing the photograph to determine whether the face mask is positioned on a face of the user.

9. The computer-implemented method of claim 1, wherein communicating with the sensor to determine whether the user is wearing the face mask includes detecting a proximity of the face mask to a face of the user using a wearable device.

10. The computer-implemented method of claim 1, wherein communicating with the sensor includes continuously or periodically communicating with the sensor to ascertain if the user is proximate or wearing the face mask over a desired period of time.

11. A system comprising:
a computing system comprising one or more processors and a data storage system in communication with the one or more processors, wherein the data storage system comprises instructions thereon that, when executed by the one or more processors, causes the one or more processors to:
detect that a user is departing from a home location;
based on the detection, initiate communication with a face mask, wherein the face mask comprises:
a material configured to cover a mouth and nose of the user;
one or more elastic bands attached to the material; and
a sensor embedded in or attached to the material, the sensor configured to provide a response when queried by the computing system;
determine whether the user is in proximity to the face mask based on the communication with the sensor;
provide an alert based on a determination that the face mask is not in proximity to the user upon departing from the home location;
communicate with the sensor to determine whether the user is wearing the face mask;
transmit a request to determine a location of the user;
query a datastore to determine whether the location is affiliated with an entity that offers rewards to users wearing face masks; and
automatically update a user profile of the user to indicate a reward is available to the user based on a determination that the user is wearing the face mask at the location of the entity that offers rewards to users wearing face masks.

12. The system of claim 11, comprising updating the user profile to provide points to the user based on the determination that the user is wearing the face mask at the location of the entity that offers rewards to users wearing face masks.

13. The system of claim 11, comprising updating the user profile to provide cash back from a purchase to the user based on the determination that the user is wearing the face mask at the location of the entity that offers rewards to users wearing face masks.

14. The system of claim 11, comprising updating the user profile to provide a discount on a purchase to the user based on the determination that the user is wearing the face mask at the location of the entity that offers rewards to users wearing face masks.

15. The system of claim 11, comprising updating a user profile of the user to indicate a reward is available to the user based on the determination that the user is in proximity to the face mask at the location of the entity that offers rewards to users wearing face masks.

16. The system of claim 11, wherein communicating with the sensor to determine whether the user is in proximity with face mask includes communicating with the sensor using a user device.

17. A non-transitory computer-readable storage medium, the non-transitory computer-readable storage medium including instructions that, when executed by computers, cause the computers to perform operations of:
detecting that a user is departing from a home location;
based on the detection, initiating communication with a face mask, wherein the face mask comprises:
a material configured to cover a mouth and nose of the user;
one or more elastic bands attached to the material; and
a sensor embedded in or attached to the material, the sensor configured to provide a response when queried by the computers;
determining whether the user is in proximity to the face mask based on the communication with the sensor;
providing an alert based on a determination that the face mask is not in proximity to the user upon departing from the home location;
communicating with the sensor to determine whether the user is wearing the face mask;
transmitting a request to determine a location of the user;
querying a datastore to determine whether the location is affiliated with an entity that offers rewards to users wearing face masks; and
automatically updating a user profile of the user to indicate a reward is available to the user based on a determination that the user is wearing the face mask at the location of the entity that offers rewards to users wearing face masks.

18. The non-transitory computer-readable storage medium of claim 17, comprising using a geolocation feature of a user device to determine the location of the user.

19. The non-transitory computer-readable storage medium of claim 17, comprising communicating with a beacon to determine the location of the user.

20. The non-transitory computer-readable storage medium of claim 17, wherein querying a datastore to determine whether the location is affiliated with an entity that offers rewards to users wearing face masks includes using a look-up table of participating entities.
